(19) United States
(12) Patent Application Publication
(10) Pub. No.: US 2026/0216011 A1
(43) Pub. Date: Jul. 30, 2026

(54) COMPOSITIONS INCLUDING SILOXYSILICATE RESIN, POLYORGANOSILOXANE COPOLYMER AND AQUEOUS PHASE FILM FORMER

(71) Applicant: L'OREAL, PARIS (FR)

(72) Inventors: Yixin YANG, UNION, NJ (US); Treasure BELLAMY, COLONIA, NJ (US); Brady ZARKET, EDISON, NJ (US)

(73) Assignee: L'OREAL, PARIS (FR)

(21) Appl. No.: 19/037,521

(22) Filed: Jan. 27, 2025

(51) Int. Cl. A61K 8/06; A61K 8/04; A61K 8/81; A61K 8/89; A61Q 19/00

(52) U.S. Cl. CPC A61K 8/062; A61K 8/042; A61K 8/8182; A61K 8/89; A61Q 19/00; A61K 2800/30

(57) ABSTRACT

The invention relates to compositions including (a) water, (b) at least one oil, (c) at least one siloxysilicate resin, (d) at least one polyorganosiloxane copolymer; and (e) at least one copolymer comprising a vinyl pyrrolidone monomer; and method of using said compositions.

### FIELD OF THE INVENTION

The present invention relates to oil-in-water (o/w) emulsions (compositions) comprising at least one siloxysilicate resin, at least one polyorganosiloxane copolymer; and at least one copolymer comprising a vinyl pyrrolidone monomer, as well as to methods and kits comprising such emulsion compositions in container(s) or in application. The compositions have beneficial cosmetic properties including, for example, as primer or setter compositions for good or improved wear, water and/or sebum resistance, and/or transfer resistance properties of separately applied compositions.

### DISCUSSION OF THE BACKGROUND

At the present time on the market for caring for and making up keratin materials, many products claim staying power throughout the day, withstanding external factors such as water, sebum, mechanical friction, etc. (waterproof mascara, food-proof lipsticks, long-lasting foundations). Long-lasting products for the lips, the eyelashes, the eyebrows or the face, which can be used at home, are mainly based on synthetic coating polymers in the presence of solvent.

One particular application, makeup “setting” is traditionally accomplished by spraying a composition including a water-soluble polymer over makeup that had been previously applied to e.g., the face. Traditional makeup setters, however, often do not greatly improve the wear of the of the underlying makeup and its resistance to external forces such as mechanical friction, water, sweat and perspiration, sebum and/or oil. The instant inventors have recognized the need for improved materials and compositions in this regard.

### SUMMARY OF THE INVENTION

The present invention relates to oil-in-water emulsion compositions including (a) water, (b) at least one oil, (c) at least one siloxysilicate resin, (d) at least one polyorganosiloxane copolymer; and (e) at least one copolymer comprising a vinyl pyrrolidone monomer. The (c) at least one siloxysilicate resin may be a trimethylsiloxysilicate. The (d) at least one polyorganosiloxane copolymer may be a silicone-modified amide. The (e) may be a cyclic amide-containing polymer having at least one of cyclic amine and acrylamide functionality. The composition may be in the form of a gel.

The composition may further include (f) at least one compound selected from the group consisting of at least one gelling agent, at least one colorant, at least one active agent, and mixtures thereof.

The (c) at least one siloxysilicate resin, (d) at least one polyorganosiloxane copolymer; and (e) at least one copolymer comprising a vinyl pyrrolidone monomer may present in a sum of total concentrations by weight in the composition of at least about 9%, such as from about 9% to about 15%. The copolymer comprising a vinyl pyrrolidone monomer (e.g. cyclic amide-containing polymer having at least one of cyclic amine and acrylamide functionality) may be present in a concentration by weight in the composition of at least 0.5%.

The water may be present in a concentration by weight in the composition from about 30% to about 90%. Furthermore, the composition may include a C2-C5 monoalcohol in a concentration by weight in the composition from about 2.5% to about 25%. According to certain other embodiments, the composition includes a plurality of or even all of the limitations specified above.

According to another aspect of the invention, a method of making up skin includes applying the above-described composition to skin having a makeup film formed thereon, wherein the composition comprises (a) water, (b) at least one oil, (c) at least one siloxysilicate resin, (d) at least one polyorganosiloxane copolymer; and (e) at least one copolymer comprising a vinyl pyrrolidone monomer.

It is to be understood that both the foregoing general description and the following detailed description are exemplary and explanatory only and are not restrictive of the invention.

### DETAILED DESCRIPTION OF THE INVENTION

In the following description of the invention and the claims appended hereto, it is to be understood that the terms used have their ordinary and accustomed meanings in the art, unless otherwise specified.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. Unless otherwise indicated, all concentrations shown as percentages are concentrations by weight and also, unless otherwise indicated, relate to the entire cosmetic adhesive composition as a whole.

“A” or “an” as used herein means “at least one.”

“At least one” means one or more and thus includes individual components as well as mixtures/combinations.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

“Film-former”, “film-forming polymer” or “film-forming agent” as used herein means a polymer or resin that leaves a continuous film on the substrate to which it is applied, for example, after a solvent accompanying the film former has evaporated, absorbed into and/or dissipated on the substrate. Such film formers may, for example be aqueous (water soluble or water-dispersible or “water phase”) film formers or “oil phase film formers.”

“Wax” as used herein is a lipophilic fatty compound that is solid at ambient temperature (25° C.) and changes from the solid to the liquid state reversibly, having a melting temperature of more than 30° C. and, for example, more than 45° C., and a hardness of more than 0.5 MPa at ambient temperature.

“Surfactant” and “emulsifier” are used interchangeably throughout this specification.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 115° C.

“Non-volatile”, as used herein, means having a flash point of greater than about 115° C.

“Polymer” as used herein means a compound which is made up of at least two monomers.

“Matte” in compositions as used herein refers to compositions having little to no light reflection. For example, matte compositions can have average gloss properties, measured at 60°, of less than or equal to 10, for example 9, preferably less than or equal to 8, 6, 5, 4 or 1, including all ranges and subranges therebetween such as 1-10, 1-5, 2-10, 3-8, less than 1, less than 3, less than 5, etc. Such measurements can be made by depositing films to be tested onto a substrate (ex. a black scrub panel P121-10N or opacity card BYK No. 2810) using a drawdown bar (for example, of 1 mil, 3 mil, or 6 mil thickness) and an Automatic Drawdown Machine. The films can then be dried at room temperature and analyzed using a gloss meter (BYK: micro-TRI-gloss) at an angle of 60°.

“Free” or “substantially free” or “devoid of” as it is used herein means that while it is preferred that no amount of the specific component be present in the composition, it is possible to have very small amounts of it in the compositions of the invention provided that these amounts do not materially affect at least one, preferably most, of the advantageous properties of the compositions of the invention. Thus, for example, “free of colorant” means that an effective amount (that is, more than trace amounts) of colorant is omitted from the composition, “substantially free of colorant” means that colorant is present in amounts not greater than 0.1% by weight, and “devoid of colorant” means that colorant is present in amounts not greater than 0.25% by weight, based on the total weight of the composition. The same nomenclature applies for all other ingredients identified throughout the application.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to keratin materials such as lips and evaluating the color of the composition after an extended period of time. For example, the color of a composition may be evaluated immediately following application to keratin materials such as lips and these characteristics may then be re-evaluated and compared after a certain amount of time. Further, these characteristics may be evaluated with respect to other compositions, such as commercially available compositions.

“Keratinous material” or “keratin material” means natural lips, skin such as the face, the body, the hands, and the area around the eyes, and keratin fibres such as eyelashes, and eyebrows.

“Physiologically acceptable” means compatible with keratinous material and having a pleasant color, odor and feel, and which does not cause any unacceptable discomfort (stinging or tautness) liable to discourage a consumer from using the composition. Compositions of the present invention may be in the form of a gel composition.

“Gel composition” means a composition which does not flow similar to a liquid when applied to a substrate, a composition which has a 3-dimensional network that inhibits the composition from spreading on, or dripping from, a substrate after application owing to gravity over a short period of time (e.g., less than 10 seconds). G′ (storage modulus) is higher than G″ (loss modulus) at all range of strains up to 300% strain. Preferably, G′ (storage modulus) is higher than G″ (loss modulus) at low strain, but with G′ decreasing and G″ increasing, and the gel has the crossover point at >0.1% strain, preferably >1% strain, and preferably less than 200% strain, preferably less than 150% strain.

“Gel Crossover Point” (Sol/Gel Point), means the point at which the G″ (loss modulus) intersects the G′ (storage modulus), reported in % strain. It is the point at which a composition goes from a more solid state to a more liquid state. An example of a method for determining gel crossover point is as follows: G″ (loss modulus) and G′ (storage modulus) using a Discovery HR-3 Rheometer by TA Instruments, having 40 mm parallel plate geometry on a stainless steel flat peltier plate. The test can be run @ 25° C., with test parameter of angular frequency of 1.0 rad/s and logarithmic sweep: Strain % 0.01 to 1000.0%, 5 points per decade. Results reported in % strain.

“Natural compound” refers to any compound derived directly from a natural substance such as a plant without having undergone any chemical modification.

“Compound of natural origin” refers to any compound derived from a natural compound which has undergone one or more chemical modifications, for example by organic synthesis reaction, without the properties of the natural compound having been modified.

“Synthetic compound” refers to any compound which is not a natural compound or a compound of natural origin.

“Room temperature” means 25° C.

“Atmospheric pressure” means 760 mmHg, i.e. 105 pascals.

The compositions and methods of the present invention can comprise, consist of, or consist essentially of the essential elements and limitations of the invention described herein, as well as any additional or optional ingredients, components, or limitations described herein or otherwise useful. For example, the aqueous phase solvent system can “consist essentially of” water and C2-C5 monoalcohol, and/or the film forming system of the composition can consist essentially of the siloxysilicate resin, polyorganosiloxane copolymer, and copolymer comprising a vinyl pyrrolidone monomer; similarly, the oil phase film forming system can consist essentially of the siloxysilicate resin, and polyorganosiloxane copolymer.

For purposes of the present invention, the “basic and novel property” associated with compositions, components and methods which “consist essentially of” identified ingredients or actions is transfer resistance.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference) number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

All U.S. patents or patent applications disclosed herein are expressly incorporated by reference in their entirety.

### Aqueous Phase Film Former

According to the present invention, compositions including one or more aqueous phase film-forming polymers (“first film-forming polymers” or “first film formers”) that is/are non-ionic water-soluble or water dispersible polymer are provided. For clarity, by “non-ionic water-soluble or water-dispersible” it is meant that the polymer is non-ionic. The polymer is also water-soluble or water-dispersible, particularly to the extent that it can be readily stabilized throughout water present in the composition.

The one or more of these first film-forming polymers are part of an aqueous phase film-forming system. The aqueous phase film forming system can comprise, consist essentially of, or consist of one aqueous phase film former.

Examples of aqueous phase film forming polymer include, but are not limited to, homopolymers and copolymers at least one vinyl (e.g. vinylpyrrolidone) monomer such as polyvinylpyrrolidone, polyvinyl alcohol, vinyl pyrrolidone/vinyl acetate copolymers, vinyl pyrrolidone/acrylic acid copolymers, vinyl pyrrolidone/acrylate copolymers, alkyl (e.g., butyl) acrylate/hydroxyalkyl (e.g., propyl)dimethicone acrylate copolymers, styrene/acrylates/ammonium methacrylate copolymers, acrylamide/sodium acryloyldimethyltaurate copolymer, etc.; water-soluble polysaccharides such as pectin, unmodified pullulan or cellulose compounds; polyurethanes; latexes; and mixtures thereof. According to certain embodiments, the aqueous phase film forming polymer is selected from a group consisting of polyvinylpyrrolidone, vinylpyrrolidone/vinyl acetate copolymer, vinylpyrrolidone/acrylic acid copolymer, polyvinyl alcohol, copolymer of N-vinyl pyrrolidone, methacrylamide, and N-vinylimidazole. According to certain other embodiments, the aqueous phase film forming polymer is a copolymer comprising a vinyl pyrrolidone monomer (and one or more second monomers). The one or more second monomers may include, for example cyclic amides, cyclic amines, (meth)acrylate, (meth)acrylamide, or other monomers different from vinylpyrrolidone.

According to preferred embodiments, the at least one aqueous phase film forming polymer is at least one cyclic amide-containing polymer. Preferably, the at least one cyclic amide-containing polymer also has at least one of cyclic amine and/or acrylamide functionality as well, preferably both of these, although it is possible for the at least one polymer to contain neither cyclic amine nor acrylamide functionality.

Cyclic amide and cyclic amine monomers useful in the preferred first film-forming polymers include those having one or more aromatic or aliphatic ring structures. These rings may have sizes ranging from about having sizes of, for example, 5 to 8 ring members.

In certain embodiments, monomers useful in forming these first film-forming polymers are polymerizable, ethylenically-unsaturated monomers having a cyclic amine residue or a cyclic amide residue. Accordingly, the cyclic amide monomers of these first film-forming polymers may include cyclic amide residues that are or include heterocyclic ring structures such as lactams and the like. These may include α-Lactam, β-lactam, γ-lactam, ö-lactam, and ε-lactam. In one preferred embodiment, the cyclic amide is a pyrrolidone (a γ-lactam), in particular vinylpyrrolidone

Useful cyclic amine residues may include any of various heterocyclic amines such as azoles, pyrroles, pyrrolidines, carbamates, and the like. In one preferred embodiment, the cyclic amine residue is an imidazole.

In certain embodiments, acrylamide monomers useful in these first film-forming polymers include those having —C3H5NO functional groups. Examples include (meth)acrylamides.

As noted above, at least one cyclic amide-containing polymer preferably has at least one of cyclic amine and acrylamide functionality as well, preferably both, although it is possible that the polymer contains neither functionality. For example, polymer(s) containing cyclic amide residue(s) but not cyclic amine or acrylamide functionality include but are not limited to vinylpyrrolidone homopolymers (polyvinylpyrrolidone) and vinylpyrrolidone copolymers containing at least one monomer other than vinylpyrrolidone such as, for example, substituted or unsubstituted C2 (acrylic acid) or C3 (allyl) groups, possibly in the form of esters (for example, acrylates or methacrylates) or ethers.

In certain embodiments, the at least one aqueous phase film forming polymer has a weight average molecular weight in a range from about 10,000 daltons to about 1,000,000 daltons, including all ranges and subranges therebetween such as, for example, 50,000 daltons to 500,000 daltons, 75,000 daltons to 300,000 daltons, and 100,000 daltons to 250,000 daltons.

In certain embodiments, the at least one cyclic amide-containing polymer is a copolymer of N-vinyl pyrrolidone, methacrylamide, and N-vinylimidazole.

In certain embodiments, the at least one cyclic amide-containing polymer may be a commercially available variety, such as LUVISET CLEAR AT3, commercially available from BASF of Ludwigshafen, Germany.

According to preferred embodiments, the compositions of the present invention are devoid of, free of, or substantially free of, polyurethanes.

Preferably, the at least one aqueous phase film forming polymer, and in particular copolymer comprising a vinyl pyrrolidone monomer is present in the compositions of the present invention in an amount of at least about 0.5% by weight, such in an amount ranging from about 0.5%, 1% or 2% to about 2%, 3%, 4%, 5%, 6%, 7%, 8%, 9%, or 10%, all weights being based on the weight of the composition as a whole.

### Siloxysilicate Resin

In accordance with the present invention, compositions comprising at least one siloxysilicate resin are provided.

The siloxysilicate resin may be a siloxysilicate resin as described for example, in U.S. Pat. Nos. 5,505,937, 5,911,974, 5,965,112, 5,985,298, 6,074,654, 6,780,422, 6,908,621, the disclosures of which are hereby incorporated by references. The at least one siloxysilicate resin is preferably an oil phase film former.

One non-limiting example of a siloxysilicate in accordance with the present invention is trimethylsiloxysilicate, which may be represented by the following formula:

[(CH₃)₃SiO]ₓ(SiO_{4/2})_{y}
wherein x and y may, for example, range from 50 to 80. Such siloxysilicates are commercially available from Dow Corning, Wacker, Milliken, Siltech, Grant industries, Momentive and Shin-Etsu Silicones, such as under the tradename Resin MQ®.

According to another embodiment of this invention, the compositions may contain silsesquioxane resins, including comprise at least one polypropyl silsesquioxane film forming resin.

Silsesquioxane resins are a specific form of silicone resin. Silicone resin nomenclature known in the art as “MDTQ” nomenclature, whereby a silicone resin is described according to the various monomeric siloxane units which make up the polymer. Each letter of “MDTQ” denotes a different type of unit. When the film forming resin is made up predominantly of tri-functional units (or T units), it is generally called a silsesquioxane resin, which is described, for example in US 2006/0292096, herein incorporated by reference.

Examples of silsesquioxane resins that may be used in the present invention are alkyl silsesquioxane resins that are silsesquioxane homopolymers and/or copolymers having an average siloxane unit of the general formula R¹ ₙSiO_{(4-n)/2}, wherein each R1 is a propyl group, wherein more than 80 mole % of R1 represent a C3-C10 alkyl group, n is a value of from 1.0 to 1.4, and more than 60 mole % of the copolymer comprises R¹SiO_{3/2} units. As each R¹ is a propyl group these polymers are called polypropylsilsesquioxane resins or “t-propyl” silsesquioxane resins. These resins and methods of making them are described, for example in U.S. Pat. Nos. 8,586,013, 2012/0301415, 2007/0093619, and 2006/0292096, all of which are herein incorporated by reference.

A non-limiting example of a polypropylsilsesquioxane resin suitable for se in the present invention is commercially available from Dow Corning as Dow Corning 670 Fluid or Dow Corning 680 Fluid. These Dow Corning resins have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value from 1.0 to 1.4, more than 60 mole % of the copolymer comprises RSiO_{3/2} units, and having a hydroxyl or alkoxy content from 0.2 to 10% by weight, for example between 1 and 4% by weight, preferably between 5 and 10% by weight, and more preferably between 6 and 8% by weight. Preferably, the polypropylsilsesquioxane resin has a molecular weight from about 5000 to about 30,000 and a Tg from about −5° C. to about 5° C.

The at least one siloxysilicate resin is generally present in the cosmetic composition of the present invention in an amount ranging from about 1% to about 15% such as from about 1%, 2%, 3%, 4% or 5% to about 7%, 8%, 10% 12% or 15%, all weights being based on the weight of the composition as a whole.

### Polyorganosiloxane Copolymer

The cosmetic compositions of the present invention also comprise at least one polyorganosiloxane-containing polymer. The at least one polyorganosiloxane-containing polymer is preferably an oil phase film former.

The polyorganosiloxane-containing polymer useful herein is a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. Non-limiting examples of polyorganosiloxane-containing polymers are disclosed, for example in U.S. Pat. No. 8,945,525, the disclosure of which is hereby incorporated by reference.

Additional polyorganosiloxane-containing polymers which may be used in the composition of the invention include silicone-modified amides. Suitable silicone-modified amides include those described in documents U.S. Pat. Nos. 5,874,069, 5,919,441, 6,051,216, and 5,981,680, the entire contents of which are hereby incorporated by reference.

A preferred polyorganosiloxane-containing polymer for use in the present invention contain at least one moiety chosen from formula (III)

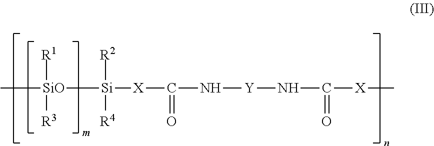

and formula (IV)

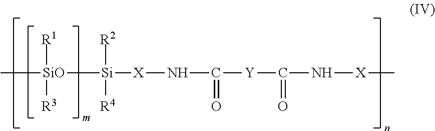

in which:
(a) R¹, R², R³ and R⁴ are the same or different and may be selected from the group consisting of methyl, ethyl, propyl, isopropyl, a siloxane chain, and phenyl;
(b) X is a linear or branched chain alkylene having 1-30 carbons;
(c) Y is selected from the group consisting of linear or branched chain alkylenes having 1-40 carbons;
(d) m is a number between 1 and 700;
(e) n is a number between 1 and 500.

Particularly preferred polyorganosiloxane-containing polymers useful herein are commercially available from Dow Corning under the tradenames DC 8178® and DC 8179®, which are known under the INCI denomination of Nylon-611/Dimethicone Copolymer.

The at least one polyorganosiloxane-containing polymer is generally present in the cosmetic composition of the present invention in an amount ranging from about 1% to about 15% such as from about 1%, 2%, 3%, 4% or 5% to about 7%, 8%, 10% 12% or 15%, all weights being based on the weight of the composition as a whole.

Furthermore, the present inventors have also found that according to certain embodiments of the invention, in order to provide sufficient haze (blurring) and optionally sufficient transfer resistance, the total concentration by weight of film-formers in the composition may be at least about 9%, such as from about 9.5% or 10% or 10.8% to about 12%, 12.1%, 12.5% or 14%

The present inventors have found that according to certain embodiments of the invention, in order to provide a combination of optimal transfer resistance and blurring/soft focus effects, the at least one siloxysilcate resin and the at least one polyorganosiloxane-containing polymer are present in a ratio by weight of siloxysilcate resin: polyorganosiloxane-containing polymer that is at least about 1.6. In certain other embodiments this ratio is from about 1.6 to about 4, such as from about 1.6 to about 3, such as from about 1.6 to about 3.

Furthermore, the present inventors have also found that according to certain embodiments of the invention, in order to provide a combination of optimal transfer resistance and blurring/soft focus effects the total concentration by weight of film-formers in the composition may be at least about 9%. In certain other embodiments, the composition exhibits both one of the above ratio requirements (siloxysilcate resin to polyorganosiloxane-containing polymer) as well as this total film former requirement. Furthermore, in order reduce potential aesthetic drawbacks from having too much film former, the total concentration of film-formers may be from about 9% to about 15% by weight, such as from about 9% or 10% to about 12% or 12.5% by weight.

In certain embodiments, the only film-formers in the composition are the (c) at least one siloxysilicate resin, (d) at least one polyorganosiloxane copolymer; and (e) at least one copolymer comprising a vinyl pyrrolidone monomer, in other embodiments other categories of film-formers (additional film formers) may be included. In certain embodiments these additional film formers are less than 50%, such as less than 25%, such as less than 10% of the total concentration of film-formers in the composition.

Regardless of whether (c) at least one siloxysilicate resin, (d) at least one polyorganosiloxane copolymer; and (e) at least one copolymer comprising a vinyl pyrrolidone monomer are the only film formers in the composition or not, the total concentration of (c), (d), and (e) may be at least about 9%, such as from about 9% to about 15% by weight, such as from about 9% to about 12% by weight in the composition, such as from about 9% or 10% to about 12% or 12.5% by weight.

### Aqueous Phase

According to preferred embodiments of the present invention, compositions comprising an aqueous phase solvent system comprising (i) water in an amount of at least about 10% by weight with respect to the total weight of the composition; and (ii) optionally, at least one C2-C5 monoalcohol are provided. Preferably, the composition is in the form of an emulsion with a continuous outer phase which is aqueous, preferably an oil-in-water (o/w) emulsion.

According to preferred embodiments, the compositions of the present invention comprise water. Preferably, the compositions comprise at least about 10% water by weight, preferably greater than 20% water by weight, preferably greater than about 25% water by weight, and preferably greater than about 30% water by weight, preferably in amounts ranging from about 30%, 40%, or 50% to about 50%, 60%, 70%, 80%, or 90%, all weights being based on the weight of the composition as a whole.

Suitable C2-C5 monoalcohols, if present, include ethanol, propanol, butanol, pentanol, isopropanol, isobutanol and isopentanol. Ethanol is particularly preferred.

Preferably, the C2-C5 monoalcohol(s), if present, is/are present in the compositions of the present invention in an amount ranging from about 1% to about 50%, preferably in amounts ranging from about 1%, 2.5%, 5%, or 10% or 15% to about 15%, 20%, 25%, 30%, or 50%, all weights being based on the weight of the composition as a whole.

Preferably, the aqueous phase solvent system of the compositions of the present invention consists essentially of, or consists of, water and, optionally, C2-C5 monoalcohols. Preferably, the aqueous phase solvent system is “free of,” “devoid of” or “substantially free of” solvents other than water and, optionally, C2-C5 monoalcohols.

### Oil Phase

According to the present invention, compositions comprising at least one oil are provided.

Oil means any hydrophobic substance that are liquid at ambient temperature (25° C.) and atmospheric pressure (760 mm Hg). They can generally be emulsifier into an aqueous phase using a suitable emulsifier. The term “volatile oil” is understood to mean, within the meaning of the invention, any oil capable of evaporating on contact with the skin in less than one hour, at ambient temperature and atmospheric pressure. The volatile oil is a volatile cosmetic compound, liquid at ambient temperature, having in particular a non-zero vapor pressure, at ambient temperature and atmospheric pressure, in particular having a vapor pressure ranging from 2.66 Pa to 40 000 Pa, in particular ranging from 2.66 Pa to 13 000 Pa and more particularly ranging from 2.66 Pa to 1300 Pa.

The volatile oil in accordance with the invention can be chosen from the group constituted of hydrocarbon oils, silicone oils and their mixtures.

The term “hydrocarbon oil” is understood to mean an oil containing predominantly hydrogen and carbon atoms and optionally one or more functions chosen from hydroxyl, ester, ether and carboxyl functions.

Within the meaning of the present invention, the term “silicone oil” denotes an oil comprising at least one Si—O group and more particularly an organopolysiloxane.

The volatile hydrocarbon oils which can be used in the compositions according to the invention can be chosen from branched C8-C16 alkanes.

Mention may in particular be made, as C8-C16 isoalkanes of petroleum origin (also known as isoparaffins), such as isododecane (also known as 2,2,4,4,6-pentamethylheptane or “IDD”), isodecane, isohexadecane and, for example, the oils sold under the Isopar® or Permethyl® tradenames. More preferentially, isododecane will be used.

Mention may be made, by way of example of volatile silicone oil which can be used in the invention, of volatile silicone oils, such as volatile linear or cyclic silicone oils, in particular those having a viscosity of 2 to 8 centistokes (2.10⁻⁶ to 8.10⁻⁶ m²/s) and containing in particular from 2 to 7 silicon atoms, these silicones optionally comprising alkyl or alkoxy groups containing from 1 to 10 carbon atoms. Mention may in particular be made, as volatile silicone oils which can be used in the invention, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane; and their mixtures. More preferentially, decamethylcyclopentasiloxane (D5) will be used.

Compositions of the present invention preferably comprise sufficient oil to form an oil-in-water emulsion, and having one or more oils preferably in an amount ranging from about 1%, 2%, 5%, or 10% or 15% to about 5%, 10%, 15%, 20%, 25%, 30%, 50% or 70%, where all weights being based on the weight of the composition as a whole.

Suitable oils include volatile and/or non-volatile oils. Such oils can be any acceptable oil including but not limited to silicone oils and/or hydrocarbon oils.

According to certain embodiments, the oil carrier comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |

Further, a volatile linear silicone oil may be employed in the present invention. Suitable volatile linear silicone oils include those described in U.S. Pat. No. 6,338,839 and WO2003042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other embodiments, the oil carrier comprises one or more non-silicone volatile oils and may be selected from volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isododecane, isodecane, and for example, the oils sold under the trade names of Isopar or Permethyl. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |
| Undecane | 62 |
| Tridecane | 94 |
| Isohexadecane | 96 |

The volatility of the solvents/oils can be determined using the evaporation speed as set forth in U.S. Pat. No. 6,338,839, the contents of which are incorporated by reference herein.

According to other embodiments of the present invention, the oil carrier comprises at least one non-volatile oil. Examples of non-volatile oils that may be used in the present invention include, but are not limited to, polar oils such as:
hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
synthetic oils or esters of formula R₅COOR₆ in which R⁵ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
synthetic ethers containing from 10 to 40 carbon atoms;
C8 to C26 fatty alcohols, for instance oleyl alcohol, cetyl alcohol, stearyl alcohol, and cetearly alcohol; and mixtures thereof.

Further, examples of non-volatile oils that may be used in the present invention include, but are not limited to, non-polar oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

According to preferred embodiments, the compositions of the present invention are devoid of, free of, or substantially free of, silicone oils.

According to preferred embodiments, the compositions of the present invention are devoid of, free of, or substantially free of, non-volatile oils.

According to preferred embodiments, the compositions of the present invention are devoid of, free of, or substantially free of, waxes.

### High HLB Surfactant

According to certain embodiments of the present invention, compositions may include at least one high HLB surfactant. “HLB” refers to the “hydrophilic-lipophilic balance” associated with emulsifiers/surfactants. In particular, “HLB” value relates to the ratio of hydrophilic groups and lipophilic groups in emulsifiers, and also relates to solubility of the emulsifiers. Lower HLB emulsifiers are more soluble in oils (lipophilic material) and are more appropriate for use in water-in-oil (W/O) emulsions. Higher HLB emulsifiers are more soluble in water (hydrophilic material) and are more appropriate for oil-in-water (O/W) emulsions. In the context of the present invention, “high HLB surfactant” means a surfactant having an HLB value of greater than 8. Also, in the context of the present invention, “high HLB value” means a surfactant having an HLB value greater than or equal to 8, preferably greater than or equal to 9, preferably greater than or equal to 10, preferably greater than or equal to 12, and preferably greater than or equal to 13.

Examples of surfactants include, but are not limited to, the following:
Polyethylene glycolated (PEG) and/or glycerolated esters or ethers, such as polyethylene glycolated and/or glycerolated branched or linear C8-C24 compounds, for example, Oleth-50, Oleth-20, Oleth-10, Ceteth-10, Steareth-20, Laureth-23, PEG-8 stearate, PEG-8 isostearate, PEG-20 stearate PEG-40 stearate, etc.;
sorbitan esters or ethers, such as oxyethylenated or nonoxyethylenated sorbitan mono- or polyalkyl esters or ethers, such as Polysorbate 21, Polysorbate 40, Polysorbate 80, Polysorbate 60, Polysorbate 61, sorbitan isostearate, glyceryl sorbitan isostearate, sorbitan sesquioleate, sorbitan laurate, sorbitan monopalmitate, sorbitan oleate, sorbitan trioleate, sorbitan monostearate and sorbitan tristearate, etc.;
Sugar mono- or polyalkyl esters or ethers, such as the mono- or polyalkyl esters or ethers of sugars as described in the patent U.S. Pat. No. 6,689,371. Mention may be made, for example, of methyl glucose isostearate, sucrose distearate, sucrose stearate, etc.;
Alkoxylated alkenyl succinates; and
Silicone derivatives, such as dimethicone copolyols, for example the mixture of cyclomethicone and of dimethicone copolyol sold under the name “DC 5225 C” by Dow Corning, and alkyl dimethicone copolyols, such as lauryl methicone copolyol, sold under the name “Dow Corning 5200 Formulation Aid” by Dow Corning, and cetyl dimethicone copolyol, sold under the name “Abil EM 90” by Goldschmidt, or the polyglyceryl-4 isostearate/cetyl dimethicone copolyol/hexyl laurate mixture sold under the name “Abil WE 90” by Goldschmidt.

The at least one high HLB surfactant is preferably present in the compositions of the present invention in an amount up to about 10%, such as of about 0.05% to about 5%, preferably from about 0.05% to about 4%, and preferably from about 0.05% to about 3%, preferably from about 0.05% to about 1% by weight with respect to the total weight of the composition, including all ranges and subranges therebetween.

### Gelling Agent/Colorant/Active Agent

### Gelling Agent

According to preferred embodiments of the present invention, compositions further comprising at least one gelling agent are provided. Suitable gelling agents include any gelling agent, such as for example, gums, clays and acrylic acid (co) polymers, such as high molecular weight homo- or co-polymers comprising acrylic acid, optionally crosslinked with a polyalkenyl polyether, including some polymers identified as “carbomer” as well as amphiphilic polymers. Preferably, the gelling agent is a gum, an amphiphilic polymer or a clay, or a combination of any of these. According to certain embodiments, the gelling agent is a crosslinked polymer.

According to preferred embodiments, the at least one gelling agent is preferably at least one gum. Suitable examples of thickening gums include, but are not limited to, Suitable gums include xanthan, sclerotium, pectin, karaya, arabic, gelatin, agar, guar, carrageenan, alginate and combinations thereof.

According to preferred embodiments, the at least one gelling agent is preferably at least one amphiphilic polymer, comprising at least one ethylenically unsaturated monomer, preferably containing a sulphonic group, in freeform or partially or totally neutralized form.

The amphiphilic polymers may comprise at least one hydrophobic portion. The hydrophobic portion present in these polymers preferably contains from 6 to 50 carbon atoms, preferably from 6 to 22 carbon atoms, preferably from 6 to 18 carbon atoms and preferably from 12 to 18 carbon atoms, including all ranges and subranges therebetween.

The amphiphilic polymers may have a molar mass ranging from 50,000 g/mole to 10,000,000 g/mole, preferably from 80,000 g/mole to 8,000,000 g/mole, and preferably from 100,000 g/mole to 7,000,000 g/mole.

The amphiphilic polymers may be based on at least one ethylenically unsaturated hydrophilic monomer A and on at least one hydrophobic monomer B. Preferably, the monomer A comprises a strong acid function, in particular a sulphonic acid or phosphonic acid function. The hydrophobic monomer B comprises at least one hydrophobic radical, chosen from: saturated or unsaturated C₆-C₁₈ linear alkyl radicals (for example, n-hexyl, n-octyl, n-decyl, n-hexadecyl, n-dodecyl or oleyl); branched alkyl radicals (for example, isostearic) or cyclic alkyl radicals (for example, cyclododecane or adamantane); C₆-C₁₈ fluoro or alkylfluoro radicals (for example, the group of formula —(CH₂)₂—(CF₂)₉—CF₃); a cholesteryl radical or radicals derived from cholesterol (for example, cholesteryl hexanoate); aromatic polycyclic groups, for instance naphthalene or pyrene; and silicone or alkylsilicone or alkylfluorosilicone radicals. Among these radicals, linear and branched alkyl radicals are preferred.

The amphiphilic polymers may be water-soluble or water-dispersible in neutralized form.

The amphiphilic polymers may be crosslinked. The crosslinking agents may be chosen from, for example, the polyolefinically unsaturated compounds commonly used for crosslinking polymers obtained by free-radical polymerization. According to one preferred embodiment of the invention, the crosslinking agent is chosen from methylenebisacrylamide, allyl methacrylate or trimethylolpropane triacrylate (TMPTA). The degree of crosslinking preferably ranges from 0.01 mol % to 10 mol %, and preferably from 0.2 mol % to 2 mol %, relative to the polymer, including all ranges and subranges therebetween.

The amphiphilic polymers may be homopolymers or copolymers.

The amphiphilic polymers can be partially or totally neutralized with a mineral base (for example, sodium hydroxide, potassium hydroxide or aqueous ammonia) or an organic base such as monoethanolamine, diethanolamine, triethanolamine, aminomethylpropanediol, N-methylglucamine, or basic amino acids, for instance arginine and lysine, and mixtures thereof.

The amphiphilic polymers may be water-soluble or water-dispersible homopolymers such as, for example, optionally cross-linked polymers of sodium 2-acrylamido-2-methylpropane sulfonate acid such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the tradename HOSTACERIN AMPS@ by Clariant.

The amphiphilic polymers may be chosen from crosslinked or non-crosslinked amphiphilic polymers of 2-acrylamido-2-methylpropanesulphonic (AMPS) acid and of at least one ethylenically unsaturated monomer comprising at least one hydrophobic portion containing from 6 to 30 carbon atoms, preferably from 6 to 22 carbon atoms, preferably from 6 to 18 carbon atoms and preferably from 12 to 18 carbon atoms, including all ranges and subranges therebetween.

Suitable examples of amphiphilic polymers include, but are not limited to, hydrophobically-modified sulfonic acid copolymers such as Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant) (crosslinked ethoxylated AMPS/behenyl methacrylate), Ammonium Acryloyldimethyltaurate/Steareth-25 Methacrylate Crosspolymer (Aristoflex HMS) (ethoxylated copolymer of AMPS/stearyl methacrylate crosslinked with trimethylol triacrylate), Aristoflex SNC (crosslinked ethoxylated AMPS/C16-C18), Aristoflex LNC (noncrosslinked AMPS/C12-C14), acrylamide/sodium acryloyldimethyl taurate copolymer/isohexadecane/polysorbate 80) (Simulgel 600) and mixtures thereof.

According to preferred embodiments, the gelling agent(s) may be at least one clay. Among the clays, mention may be made of clays of the smectite family, such as laponite, of the kaolinite family, such as kaolinite, dickite, nacrite, optionally modified clays of the halloysite, dombassite, antigorite, berthierine, pyrophyllite, montmorillonite, beidellite, vermiculite, talc, stevensite, hectorite, saponite, chlorite, sepiolite and illite family.

Clays are products that are already well known per se, which are described, for example, in the publication “Mineralogie des argiles” [“Clay Mineralogy”], S. Caillere, S. Henin, M. Rautureau, 2nd Edition 1982, Masson, the teaching of which is included herein by way of reference.

Natural clay is a sedimentary rock composed to a large extent of specific minerals, silicates generally of aluminium. Kaolin is thus a natural clay.

The clays may also be synthetic. Thus, Sumecton mentioned below is a synthetic saponite.

Preferably, in the context of the present invention, use is made of clays that are cosmetically compatible and acceptable with keratin materials. Clays that may especially be mentioned include kaolinite, montmorillonites, saponites, laponites, hectorites, and illites. Mixtures of clays and natural clays may also be used.

Natural clays that may be mentioned include green clays, in particular rich in illite; clays rich in montmorillonite, known as fuller's earth, or such as bentonite or else white clays rich in kaolinite. As bentonites, mention may in particular be made of those sold under the names Bentone 38 VCG®, Bentone Gel CAO V®, Bentone 27 V® and Bentone Gel MIO VR by the company Elementis.

By way of clay rich in montmorillonite, mention may be made of the aluminium silicate hydrate sold under the name Gel White HR by the company Rockwood.

By way of saponite, which belongs to the montmorillonite family, mention may be made of synthetic saponite, in particular the product sold by the company Kunimine under the name Sumecton®.

By way of tack, mention may be made of those sold under the names Rose Talc® and Talc SG-2000@ sold by the company Nippon Talc, Luzenac Pharma M® sold by the company Luzenac, J-68BC® from US Corporation and Micro ACE-P-3® sold by the company Nippon Talc.

Preferably, if present, the gelling agent(s) is/are present in the compositions of the present invention in amounts ranging from about 0.1% or 0.2% to about 0.5%, 1%, 2%, 2.5%, or 5% by weight, all weights based on the weight of the composition as a whole.

### Colorant (Coloring Agent)

According to embodiments of the present invention, compositions optionally further comprising at least one coloring agent are provided. According to preferred embodiments, the at least one coloring agent, if present, is at least one surface-treated pigment. “Surface-treated pigment” means pigments that have totally or partially undergone a surface treatment of chemical, electronic, electrochemical, mechanochemical or mechanical nature, with a surface treatment agent. Preferably, the pigments are selected from inorganic pigments or inorganic/organic mixed pigments.

Surface treatment agents may be selected from the group consisting of alkyl silanes, organotitanates, halogenated phosphonates, and halogenated organosilanes. According to preferred embodiments, pigments have been surface treated with a surface treatment agent selected from the group consisting of alkoxylated alkyl silanes such as, for example, ethoxylated and/or propoxylated C2-C8 alkyl silanes, and salts thereof, organotitanates such as, for example, titanium salts of fatty acids such as, for example, C2-C8 alkylated titanium salts of C9-C24 fatty acids such as stearic acid, isostearic acid, oleic acid, cetearic acid, cetyl acid, etc., halogenated organophosphonates such as, for example, perfluoroalkyl phosphonates, and salts thereof, and halogenated organosilanes such as, for example, perfluoro C2-C8 alkyl silanes (optionally ethoxylated and/or propoxylated), and salts thereof. Specific examples of suitable surface treatment agents include (1)triethoxy caprylylsilane, (2) perfluorooctyltriethoxysilane, (3) sodium perfluorohexylethylphosphonate and (4) isopropyl titanium triisosterate.

Preferred surface treatment agents are selected from the group consisting of alkyl silanes and halogenated organosilanes. According to preferred embodiments, pigments have been surface treated with a surface treatment agent selected from the group consisting of alkoxylated alkyl silanes such as, for example, ethoxylated and/or propoxylated C2-C8 alkyl silanes, and salts thereof, and halogenated organosilanes such as, for example, perfluoro C2-C8 alkyl silanes (optionally ethoxylated and/or propoxylated), and salts thereof. Specific examples of suitable surface treatment agents include (1) triethoxy caprylylsilane, and (2) perfluorooctyltriethoxysilane.

The surface-treated pigments of the present invention can be prepared according to surface treatment techniques well known to a person of ordinary skill in the art or can be found commercially.

For example, the surface treatment agent with which the pigments are treated can be deposited on the pigments by solvent evaporation, chemical reaction between the molecules of the surface treatment agent or creation of a covalent bond between the surface treatment agent and the pigments. The surface treatment can thus be carried out, for example, by chemical reaction of a surface treatment agent with the surface of the pigments and creation of a covalent bond between the surface treatment agent and the pigments. An exemplary method is described, for example, in U.S. Pat. No. 4,578,266, the entire contents of which is hereby incorporated by reference.

The at least one surface-treated pigment, if present, preferably is present in the compositions of the present invention in an active solid content amount ranging from about 1% to about 30%, preferably from about 3% to about 25%, and preferably from about 5% to about 20%, by weight with respect to the total weight of the composition, including all ranges and subranges there between.

According to preferred embodiments of the present invention, compositions further comprising at least one unsurfaced-treated (not surface-treated) coloring agent are provided. Such coloring agents, if present, may be in addition to, or instead of, the surface-treated pigment discussed above.

According to this embodiment the coloring agent is preferably chosen from pigments which are not surface-treated, dyes, such as liposoluble dyes, nacreous pigments, and pearling agents.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 40% by weight of the total weight of the composition, such as from 0.0001% to 30%, including all ranges and subranges therebetween.

The nacreous pigments which may be used according to the present invention may be chosen from colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, be present in the composition in a concentration ranging up to 50% by weight of the total weight of the composition, such as from 0.0001% to 40%, preferably from 0.001% to 30%, including all ranges and subranges therebetween.

The non-surface treated pigments, which may be used according to the present invention, may be chosen from white, colored, inorganic, organic, polymeric, and nonpolymeric pigments. Representative examples of mineral pigments include titanium dioxide, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the coloring agents may be present in the composition in a concentration ranging up to 50% by weight of the total weight of the composition, such as from 0.0001% to 40%, and further such as from 0.001% to 30%, including all ranges and subranges therebetween.

According to preferred embodiments, the compositions of the present invention are devoid of, free of, or substantially free of, coloring agents.

### Active Agent

According to preferred embodiments of the present invention, compositions further comprising at least one active agent are provided. Preferably, the active agent is in the aqueous phase (hydrophilic active agent), although active agents may be present in the oil phase (hydrophobic active agent).

In some embodiments, the active agent may be:
a moisturizing agent, such as a polyol such as, for example, glycerin and sugars, urea and its derivatives, such as in particular hydroxyalkyl urea, in particular hydroxyalkylurea, and mixtures thereof;
a dequamating agent, which may be a compound capable of acting either directly on desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol and certain jasmonic acid derivatives; or acting on the enzymes involved in the desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; aminosulfonic compounds and in particular (N-2-hydroxyethylpiperazine-N-2-ethane) sulfonic acid (HEPES); 2-oxothiazolidine-4-carboxylic acid (procysteine) derivatives; α-amino acid derivatives of the type such as glycine (as described in EP-0 852 949 and sodium methylglycinediacetate sold by BASF under the trade name Trilon M); honey; sugar derivatives such as O-octanoyl-6-D-maltose and N-acetylglucosamine;
a humectant;
an anti-aging agent, which may include, for example, one or more of C-beta-D-xylopyranoside-2-hydroxypropane (Pro-Xylane), retinol, peptides, caffeine, and other components that provide improvement to skin texture, any other suitable soluble/dispersible targeted active ingredient, and combinations thereof.
a mattifying agent, which may include, but is not limited to, mattifying fillers such as, for example, talc, silica, silicone elastomers, and polyamides, and waxes such as, for example, beeswax and copernicia cerifera (carnauba) wax.
an antimicrobial agent, non-limiting examples of which include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, micronazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanalide, octopirox, octoxyglycerine, octanoyiglycine, caprylyl glycol, 10-hydroxy-2-decanoic acid, dichlorophenylimidazole dioxolane and its derivatives, farnesol and phytosphingosines, and mixtures thereof;
a pigment modifying agent and/or skin lightening agents, such as double-stranded RNA oligonucleotides are useful for decreasing tyrosinase expression. Mention may also be made of ceramides, vitamin C and derivatives thereof, in particular vitamin CG, CP and 3-O ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSH® from Seppic;
a vitamin such as a vitamin A compound such as retinol or retinoic acid, vitamin B compound such as vitamin B1, B3 or B6, a vitamin C compound such as an ascrobyl phosphate salt (e.g., magnesium or sodium, and/or derivatives thereof;
an ultraviolet (UV) filter such as, e.g., an aminobenzoic acid derivative, a dibenzoylmethane derivative, a salicylic acid derivative, a cinnamic derivative, a β,βdiphenylacrylate derivative, a benzophenone derivative, benzylidene camphor derivative, and mixtures thereof. Mention may be made especially of ethylhexyl methoxycinnamate sold under the tradename UVINUL MC 80® by the company BASF, of ethylhexyl salicylate sold under the tradename NEO HELIOPAN OS® by the company SYMRISE and of octocrylene sold under the tradename NEO HELIOPAN 303® by the company SYMRISE; and/or a combination thereof.

According to certain embodiments, compositions of the present invention include at least one or at least two of gelling agent, colorant and active agents.

### Additional Additives

The composition of the invention can also comprise any additive usually used in the field under consideration. For example, additional film forming agents (in addition to the siloxysilicate resin, at least one polyorganosiloxane copolymer and at least one copolymer comprising a vinyl pyrrolidone monomer; waxes, dispersants such as poly(12-hydroxystearic acid), preserving agents, fragrances, fillers, antioxidants, neutralizing agents, silicone elastomers, and mixtures thereof can be added. However, it is to be understood that preferred embodiments of the present invention include compositions which are “free,” substantially free” or “devoid” of the ingredients discussed in this paragraph such as additional film forming agents and waxes.

According to preferred embodiments, the compositions of the present invention contain a total solids content (excluding film formers) of 10% by weight or less, preferably 5% by weight or less, or preferably 3% by weight or less based on the weight of the composition. Preferably, compositions of the present invention are devoid of, free of, or substantially free of, solids (excluding film formers).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Needless to say, the composition of the invention should be cosmetically or dermatologically acceptable, i.e., it should contain a non-toxic physiologically acceptable medium and should be able to be applied to keratinous material of human beings.

According to certain embodiments of the invention, the composition is a spray composition. “Spray composition” means a composition having sufficient fluidity to allow it to be used in a dispenser which delivers product in the form of a spray. For example, suitable dispensers include aerosol devices (for example, where compositions are dispensed with the aid of a propellant) or manual devices (for example, a pump device) as is known in the art. Preferably, a “spray composition” has a viscosity of less than 3,000 centipoise (cps), preferably less than 2,000 cps, and preferably less than 1,000 cps.

Suitable spray compositions include trigger sprays, pump sprays, or aerosol sprays (the latter typically using an inert propellant).

For embodiments in which the composition is a spray composition (spray compositions are discussed below), the amount of at least one gelling agent present will be selected such that it does not render the composition incapable of being a spray composition.

According to preferred embodiments of the present invention, methods of caring for, and/or making up, keratinous material by applying compositions of the present invention to the keratinous material in an amount sufficient to care for, and/or to make up, the keratinous material are provided. Similarly, preferred embodiments of the present invention include methods of priming keratinous material for a cosmetic composition and/or of setting a cosmetic composition which has been previously applied to keratinous material by applying compositions of the present invention over keratinous material prior to application of a cosmetic composition (priming) and/or by applying compositions of the present invention over skin that a had a color cosmetic layer previously formed thereon (setting). Preferably, the cosmetic composition used in conjunction with compositions of the present invention is a color cosmetic composition.

Preferably, “making up” the keratin material includes applying at least one coloring agent to the keratin material (in either the composition itself or in a color coat composition applied either over or under the composition as described above) in an amount sufficient to provide color to the keratin material.

According to particularly preferred embodiments of the present invention, methods of making up keratinous material such as skin comprising (1) applying a color cosmetic composition such as a foundation to the keratinous material (e.g., skin), (2) allowing the color cosmetic composition to dry on the keratinous material (e.g., skin), preferably allowing it to dry for about 1 minute to about 15 minutes, preferably for about 5 minutes to 10 minutes, and (3) applying a composition of the present invention over the dried color cosmetic composition which has been applied to keratinous material (e.g., skin) are provided. Preferably, compositions of the present invention are applied over the dried color cosmetic composition in an amount sufficient to improve transfer resistance of the color cosmetic composition as compared to the transfer resistance provided by the color cosmetic composition when applied to keratinous material by itself.

According to certain preferred embodiments, a method of making up the skin includes applying a composition of the present invention to skin having a makeup film formed thereon. The makeup film may result from, for example, applying a foundation or other first makeup composition such as one including a coloring agent being applied to the skin and optionally being allowed to dry, such as by waiting for a time period described in the paragraph above. The composition of the present invention includes (a) water, (b) at least one oil, (c) at least one siloxysilicate resin, (d) at least one polyorganosiloxane copolymer; and (e) at least one aqueous phase film former. In this embodiment, the composition of the present invention acts as a makeup setter.

Compositions of the present invention may be applied by spraying or rubbing with hands, a pad or an applicator.

According to preferred embodiments of the present invention, kits comprising, as separate compositions in one or more containers within the kits, (A) at least one composition of the present invention, in particular a setter or primer composition for cosmetics, including siloxysilicate resin, polyorganosiloxane copolymer; and copolymer comprising a vinyl pyrrolidone monomer.

; and (B) at least one other composition such as a cosmetic composition comprising at least one colorant are provided.

Compositions (A) and (B) may be contained in different portions or sections of the same container within the kit. However, compositions (A) and (B) may also be in different containers with the kit.

According to preferred embodiments of the present invention, sets, as applied onto keratinous material, comprising (A) at least one first layer of at least one composition comprising at least one colorant; and (B) at least one second layer of at least one composition of the present invention, in particular a setter or primer composition for cosmetics, comprising siloxysilicate resin, polyorganosiloxane copolymer; and copolymer comprising a vinyl pyrrolidone monomer.
are provided. Preferably, the at least one second layer is applied over the at least one first layer (in which case the second layer is a setter layer formed by application of a setter composition to the first layer). However, if the at least one first layer is applied over the at least one second layer, the at least one second layer is a primer layer formed by application of a primer composition over keratinous material.

According to a preferred embodiment of the present invention, compositions having improved cosmetic properties such as, for example, increased water-resistance, sebum-resistance and/or transfer-resistance are provided. Preferably, the composition is a composition for skin. Most preferably, the composition is a setter or primer composition. Also preferably, the composition further comprises at least one gelling agent and/or at least one active agent.

According to a preferred embodiment of the present invention, methods of making oil-in-water (o/w) emulsions (compositions) of the present invention comprising combining siloxysilicate resin, polyorganosiloxane copolymer and copolymer comprising a vinyl pyrrolidone monomer in the amounts and/or ratios discussed above during formation of the emulsion composition are provided. Preferably, the composition is a composition for application to skin. Most preferably, the composition is a setter or primer composition, and/or is in the form of a gel. Also preferably, the composition further comprises at least one gelling agent and/or at least one active agent.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLES

### Example 1—Sample Inventive Compositions

Example inventive compositions are given in Table 3 below.

TABLE 3

| Chemical Name/ | Total %100.00 - General | Total 100% - Preferred |
|---|---|---|
| Function | % wt/wt | % wt/wt |
| Surfactants | 0-10% | 0.1-2.5% |
| Copolymer comprising a | 0.5-10% | 1-5% |
| vinyl pyrrolidone | | |
| monomer | | |
| Siloxysilicate resin | 1-15% | 4-8% |
| Polyorganosiloxane | 1-15% | 2-7% |
| Copolymer | | |
| Oils | 1-30% | 2-10% |
| Colorant/fragrance/active | Optional | Substantially free |
| Water | QS | QS |
| C2-C5 Monoalcohol | 0-25% | 2.5-25% |
| Gelling Agent | 0.1-10% | 0.1-2.5% |

### Example 2—Testing Protocol-Transfer Resistance and Haze

(Dry) Transfer resistance can be determined as follows.

A color cosmetic composition (e.g., foundation such as L′Oreal Paris Makeup True Match Liquid Foundation) is applied on keratinous material (e.g., skin) in a controlled amount (e.g., 0.020 gram) in a controlled area (e.g., 3 cm×3 cm square). Allow 5-15 minutes until the composition has dried.

Apply a test composition (setter) in a controlled amount (e.g., the same amount as was applied of the color cosmetic composition, such as 0.020 gram) over the applied color cosmetic composition and allow it to dry.

A white cotton ball/pad swatch is then used to rub across the area an identified number of times (for example, 5 times or more) to assess transfer resistance of color cosmetic composition to the ball/pad swatch after such rubbing. Transfer resistance can be evaluated by determining the difference in the amount of color cosmetic composition transferred to the cotton ball/pad swatch, with or without the applied setting composition. Transfer resistance is evaluated on a 1 to 5 scale, with 1=minimal improvement in transfer resistance of color cosmetic composition to the cotton ball/pad swatch; 3=medium improvement in transfer resistance of color cosmetic composition to the cotton ball/pad swatch; and 5=large improvement in transfer resistance of color cosmetic composition to the ball/pad swatch, with little or no transfer of color cosmetic composition to the cotton ball/pad swatch occurring. Results for this testing are shown in tables below as “Transfer test.”

Formula film haze/blurriness level can be determined as follows.

Apply the test composition (setter) onto a clear plastic film with a fixed thickness of 6 mils (~150 micron). Allow the films to dry completely. Measure the film's haze using a haze meter via ASTM (D1003) setting (e.g., BYK Haze-Gard I, Byk Instruments located in Geretsried, Germany). Record the average haze value from three replicate measurements. Results for this testing are shown in tables below as “Haze.”

The following compositions: inventive examples Ex 1-24, as well as comparative examples C1 and C2 were prepared and tested as setter compositions for L′Oreal Paris Makeup True Match Liquid Foundation according to this protocol.

Comparative examples C1 and C2 included no siloxysilcate resin and no polyorganosiloxane copolymer but did include a copolymer comprising a vinyl pyrrolidone monomer. They were otherwise identical to the other test compositions (and q.s with isododecane).

Examples Ex 1-24 were representative of the invention compositions including siloxysilicate resin, polyorganosiloxane copolymer; and copolymer comprising a vinyl pyrrolidone monomer.

Specific test compositions and test results are shown in Tables 4, 5, 6 and 7 below.

TABLE 4

| | EX 1 | EX 2 | EX 3 | EX 4 | EX 5 | EX 6 | EX 7 | EX 8 |
|---|---|---|---|---|---|---|---|---|
| Isododecane | 5.9 | 5.9 | 5.9 | 7.34 | 7.34 | 7.34 | 7.34 | 7.34 |
| Nylon-611/Dimethicone | 2.82 | 2.82 | 2.82 | 2.54 | 2.54 | 2.54 | 3.8 | 3.8 |
| Copolymer (Pspa) | | | | | | | | |
| Trimethylsiloxysilicate | 11.28 | 11.28 | 11.28 | 10.14 | 10.14 | 10.14 | 8.88 | 8.88 |
| (MQ) | | | | | | | | |
| VP/Methacrylamide/Vinyl | 0.00 | 1.30 | 2.60 | 0.00 | 1.30 | 2.60 | 0.00 | 1.30 |
| Imidazole Copolymer | | | | | | | | |
| Acrylamide/Sodium | 0.60 | 0.60 | 0.60 | 0.60 | 0.60 | 0.60 | 0.60 | 0.60 |
| Acryloyldimethyltaurate | | | | | | | | |
| Copolymer | | | | | | | | |
| Sorbitan Oleate | 0.04 | 0.04 | 0.04 | 0.04 | 0.04 | 0.04 | 0.04 | 0.04 |
| Polysorbate 80 | 0.10 | 0.10 | 0.10 | 0.10 | 0.10 | 0.10 | 0.10 | 0.10 |
| Isohexadecane | 0.32 | 0.32 | 0.32 | 0.32 | 0.32 | 0.32 | 0.32 | 0.32 |
| Ethanol | 25.00 | 25.00 | 25.00 | 25.00 | 25.00 | 25.00 | 25.00 | 25.00 |
| Water | QS | QS | QS | QS | QS | QS | QS | QS |
| PSPA (active) | 2.82 | 2.82 | 2.82 | 2.54 | 2.54 | 2.54 | 3.8 | 3.8 |
| MQ (active) | 11.28 | 11.28 | 11.28 | 10.14 | 10.14 | 10.14 | 8.88 | 8.88 |
| Aqueous Phase Film | 0 | 1.3 | 2.6 | 0 | 1.3 | 2.6 | 0 | 1.3 |
| Former | | | | | | | | |
| Total Film Former | 14.1 | 15.4 | 16.7 | 12.68 | 13.98 | 15.28 | 12.68 | 13.98 |
| MQ/PSPA | 4 | 4 | 4 | 3.99 | 3.99 | 3.99 | 2.34 | 2.34 |
| Transfer test | 5 | 4 | 5 | 3 | 4 | 5 | 4 | 4 |
| Haze | 69.6 | 52.7 | 66.4 | 23 | 45.6 | 59.3 | 25.1 | 50.1 |

TABLE 5

| | EX 9 | EX 10 | EX 11 | EX 12 | EX 13 | EX 14 | EX 15 | EX 16 |
|---|---|---|---|---|---|---|---|---|
| Isododecane | 7.34 | 10.495 | 10.495 | 10.495 | 10.50 | 10.50 | 10.50 | 10.505 |
| Nylon-611/Dimethicone | 3.8 | 2.86 | 2.86 | 2.86 | 3.80 | 3.80 | 3.80 | 4.76 |
| Copolymer (Pspa) | | | | | | | | |
| Trimethylsiloxysilicate | 8.88 | 6.645 | 6.645 | 6.645 | 5.70 | 5.70 | 5.70 | 4.755 |
| (MQ) | | | | | | | | |
| VP/Methacrylamide/Vinyl | 2.60 | 0.00 | 1.30 | 2.60 | 0.00 | 1.30 | 2.60 | 0.00 |
| Imidazole Copolymer | | | | | | | | |
| Acrylamide/Sodium | 0.60 | 0.60 | 0.60 | 0.60 | 0.60 | 0.60 | 0.60 | 0.60 |
| Acryloyldimethyltaurate | | | | | | | | |
| Copolymer | | | | | | | | |
| Sorbitan Oleate | 0.04 | 0.04 | 0.04 | 0.04 | 0.04 | 0.04 | 0.04 | 0.04 |
| Polysorbate 80 | 0.10 | 0.10 | 0.10 | 0.10 | 0.10 | 0.10 | 0.10 | 0.10 |
| Isohexadecane | 0.32 | 0.32 | 0.32 | 0.32 | 0.32 | 0.32 | 0.32 | 0.32 |
| Ethanol | 25.00 | 25.00 | 25.00 | 25.00 | 25.00 | 25.00 | 25.00 | 25.00 |
| Water | QS | QS | QS | QS | QS | QS | QS | QS |
| PSPA (active) | 3.8 | 2.86 | 2.86 | 2.86 | 3.8 | 3.8 | 3.8 | 4.76 |
| MQ (active) | 8.88 | 6.645 | 6.645 | 6.645 | 5.7 | 5.7 | 5.7 | 4.755 |
| Aqueous Phase Film | 2.6 | 0 | 1.3 | 2.6 | 0 | 1.3 | 2.6 | 0 |
| Former | | | | | | | | |
| Total Film Former | 15.28 | 9.505 | 10.805 | 12.105 | 9.5 | 10.8 | 12.1 | 9.515 |
| MQ/PSPA | 2.34 | 2.32 | 2.32 | 2.32 | 1.5 | 1.5 | 1.5 | 1 |
| Transfer test | 5 | 4 | 3 | 3 | 2 | 2.3 | 2 | 2 |
| Haze | 68.5 | 34.1 | 86.5 | 95.3 | 27.4 | 70.7 | 93.7 | 27 |

TABLE 6

| | EX 17 | EX 18 | EX 19 | EX 20 | EX 21 | EX 22 | EX 23 | EX 24 |
|---|---|---|---|---|---|---|---|---|
| Isododecane | 10.51 | 10.51 | 10.505 | 10.505 | 10.505 | 10.51 | 10.51 | 10.51 |
| Nylon-611/Dimethicone | 4.76 | 4.76 | 5.7 | 5.7 | 5.7 | 6.64 | 6.64 | 6.64 |
| Copolymer (Pspa) | | | | | | | | |
| Trimethylsiloxysilicate | 4.76 | 4.76 | 3.795 | 3.795 | 3.795 | 2.85 | 2.85 | 2.85 |
| (MQ) | | | | | | | | |
| VP/Methacrylamide/Vinyl | 1.30 | 2.60 | 0.00 | 1.30 | 2.60 | 0.00 | 1.30 | 2.60 |
| Imidazole Copolymer | | | | | | | | |
| Acrylamide/Sodium | 0.60 | 0.60 | 0.60 | 0.60 | 0.60 | 0.60 | 0.60 | 0.60 |
| Acryloyldimethyltaurate | | | | | | | | |
| Copolymer | | | | | | | | |
| Sorbitan Oleate | 0.04 | 0.04 | 0.04 | 0.04 | 0.04 | 0.04 | 0.04 | 0.04 |
| Polysorbate 80 | 0.10 | 0.10 | 0.10 | 0.10 | 0.10 | 0.10 | 0.10 | 0.10 |
| Isohexadecane | 0.32 | 0.32 | 0.32 | 0.32 | 0.32 | 0.32 | 0.32 | 0.32 |
| Ethanol | 25.00 | 25.00 | 25.00 | 25.00 | 25.00 | 25.00 | 25.00 | 25.00 |
| Water | QS | QS | QS | QS | QS | QS | QS | QS |
| PSPA (active) | 4.76 | 4.76 | 5.7 | 5.7 | 5.7 | 6.64 | 6.64 | 6.64 |
| MQ (active) | 4.755 | 4.755 | 3.795 | 3.795 | 3.795 | 2.85 | 2.85 | 2.85 |
| Aqueous Phase Film | 1.3 | 2.6 | 0 | 1.3 | 2.6 | 0 | 1.3 | 2.6 |
| Former | | | | | | | | |
| Total Film Former | 10.815 | 12.115 | 9.495 | 10.795 | 12.095 | 9.49 | 10.79 | 12.09 |
| MQ/PSPA | 1 | 1 | 0.67 | 0.67 | 0.67 | 0.43 | 0.43 | 0.43 |
| Transfer test | 2.5 | 2.3 | 3 | 3 | 3 | 2.5 | 2.7 | 2 |
| Haze | 82.9 | 94.6 | 59.4 | 88.3 | 83.3 | 34.3 | 82.8 | 86.1 |

TABLE7

| | C 1 | C 2 |
|---|---|---|
| Isododecane | 0 | 0 |
| Nylon-611/Dimethicone | 0.00 | 0.00 |
| Copolymer (Pspa) | | |
| Trimethylsiloxysilicate | 0.00 | 0.00 |
| (MQ) | | |
| VP/Methacrylamide/Vinyl | 1.30 | 2.60 |
| Imidazole Copolymer | | |
| Acrylamide/Sodium | 0.60 | 0.60 |
| Acryloyldimethyltaurate | | |
| Copolymer | | |
| Sorbitan Oleate | 0.04 | 0.04 |
| Polysorbate 80 | 0.10 | 0.10 |
| Isohexadecane | 0.32 | 0.32 |
| Ethanol | 25.00 | 25.00 |
| Water | QS | QS |
| Aqueous Phase Film | 1.3 | 2.6 |
| Former | | |
| Total Film Former | 1.3 | 2.6 |
| Transfer Test | 2 | 1 |
| Haze | 39 | 28.5 |

As can be seen, setter compositions corresponding to the present invention resulted in transfer resistance and haze (blurring, soft focus effect) when used in conjunction with the commercial foundation product as compared to the comparative setter compositions containing no siloxysilicate resin and no polyorganosiloxane.

What is claimed is:

1. An oil-in water emulsion composition comprising (a) water, (b) at least one oil,
(c) at least one siloxysilicate resin, (d) at least one polyorganosiloxane copolymer; and
(e) at least one copolymer comprising a vinyl pyrrolidone monomer.

2. The composition of claim 1, wherein the (e) at least one copolymer comprising a vinyl pyrrolidone monomer is a cyclic amide-containing polymer having at least one of cyclic amine and acrylamide functionality.

3. The composition of claim 1 wherein the (e) at least one copolymer comprising a vinyl pyrrolidone monomer is a copolymer of N-vinyl pyrrolidone, methacrylamide, and N-vinylimidazole.

4. The composition of claim 1, in the form of a gel.

5. The composition of claim 1, wherein the composition further comprises (f) at least one compound selected from the group consisting of at least one gelling agent, at least one colorant, at least one active agent, and mixtures thereof.

6. The composition of claim 1, wherein the (c) at least one siloxysilicate resin is a trimethylsiloxysilicate.

7. The composition of claim 1, wherein the (d) at least one polyorganosiloxane copolymer is a silicone-modified amide.

8. The composition of claim 1, wherein the (c) at least one siloxysilicate resin, (d) at least one polyorganosiloxane copolymer; and (e) at least one copolymer comprising a vinyl pyrrolidone monomer are present in a sum of total concentrations by weight in the composition of at least 9%.

9. The composition of claim 1, wherein the (c) at least one siloxysilicate resin, (d) at least one polyorganosiloxane copolymer; and (e) at least one copolymer comprising a vinyl pyrrolidone monomer are present in a sum of total concentrations by weight in the composition from about 9% to about 15%.

10. The composition of claim 2, wherein the at least one copolymer comprising a vinyl pyrrolidone monomer is present in a concentration by weight in the composition of at least 0.5%.

11. The composition of claim 1, wherein the composition is devoid of colorant.

12. A method of making up skin comprising applying a composition to skin having a makeup film formed thereon, wherein the composition comprises (a) water, (b) at least one oil, (c) at least one siloxysilicate resin, (d) at least one polyorganosiloxane copolymer; and (e) at least one copolymer comprising a vinyl pyrrolidone monomer.

13. The method of claim 12 comprising applying the composition to said skin, wherein said skin had a color cosmetic layer previously formed thereon, wherein said composition serves to set the color cosmetic layer previously formed thereon.

14. The method of claim 12 wherein the (e) at least one copolymer comprising a vinyl pyrrolidone monomer is a cyclic amide-containing polymer having at least one of cyclic amine and acrylamide functionality.

15. The method of claim 12, wherein the (c) at least one siloxysilicate resin is a trimethylsiloxysilicate.

16. The method of claim 12, wherein the (d) at least one polyorganosiloxane copolymer is a silicone-modified amide.

17. The method of claim 12, wherein the (c) at least one siloxysilicate resin, (d) at least one polyorganosiloxane copolymer; and (e) at least one copolymer comprising a vinyl pyrrolidone monomer are present in a sum of total concentrations by weight in the composition of at least 9%.

18. The method of claim 12, wherein the (c) at least one siloxysilicate resin, (d) at least one polyorganosiloxane copolymer; and (e) at least one copolymer comprising a vinyl pyrrolidone monomer are present in a sum of total concentrations by weight in the composition from about 9% to about 15%.

19. The method of claim 14, wherein the at least one copolymer comprising a vinyl pyrrolidone monomer is present in a concentration by weight in the composition of at least 0.5%.

20. The method of claim 12, wherein the composition is devoid of colorant.
